(19) United States
(12) Patent Application Publication
(10) Pub. No.: US 2020/0069178 A1
(43) Pub. Date: Mar. 5, 2020

(54) IMAGE-BASED VIEWER ADJUSTMENT MECHANISM

(71) Applicant: Intuitive Surgical Operations, Inc., Sunnyvale, CA (US)

(72) Inventors: Randal P. Goldberg, San Mateo, CA (US); Heath Feather, Cupertino, CA (US); Alexandre V. Matveev, Santa Clara, CA (US)

(21) Appl. No.: 16/555,579

(22) Filed: Aug. 29, 2019

Related U.S. Application Data
(60) Provisional application No. 62/724,372, filed on Aug. 29, 2018.

(51) Int. Cl. A61B 3/11; H04N 13/383; H04N 13/344; A61B 3/00; A61B 3/113

(52) U.S. Cl. CPC A61B 3/111; H04N 13/383; A61B 3/113; A61B 3/005; H04N 13/344

(57) ABSTRACT

An apparatus includes a stereoscopic image viewer. The stereoscopic image viewer includes a housing, a first lens, a second lens, and an eye-tracking image capture unit. The first lens, the second lens, and the eye-tracking image capture unit are contained in the housing. The eye-tracking image capture unit has a field of view and the eye-tracking image capture unit being is mounted within the housing with the first lens and the second lens within the field of view.

### BACKGROUND

### Field of the Invention

The present invention relates generally to stereoscopic image viewers, and more particularly to adjusting an optical spacing of a stereoscopic image viewer to match an interpupillary distance of a user.

### Description of Related Art

A surgeon's console 114 (FIG. 1) is commonly part of a teleoperated computer-assisted medical system. A user sitting at surgeon's console 114 looks through lenses mounted in surgeon's console 114 to view a stereoscopic image of a site of interest. Surgeon's console 114 includes a user manipulated mechanical mechanism that allows a user to adjust a spacing between the lenses of the stereoscopic image viewer to match the user's interpupillary distance.

Similarly, a head mounted display device includes lens used to view a stereoscopic image. Various mechanisms have been developed to facilitate viewing a stereoscopic image. See, for example, U.S. Pat. No. 9,600,068 B2 (issued on Mar. 21, 2017, disclosing “Digital Inter-Pupillary Distance Adjustment”) and U.S. Patent Application Publication No. US 2017/0344107 A1 (filed on May 25, 2016, disclosing “Automatic View Adjustments for Computing Devices Based on Interpupillary Distances Associated with their Users”).

### SUMMARY

In one aspect, an apparatus includes a stereoscopic image viewer. The stereoscopic image viewer includes a housing, a first lens, a second lens, and an eye-tracking image capture unit. The first lens, the second lens, and the eye-tracking image capture unit are contained in the housing. The eye-tracking image capture unit has a field of view and the eye-tracking image capture unit is mounted within the housing with the first lens and the second lens within the field of view. In this aspect, the first and second lenses are completely within the field of view.

In this apparatus, the stereoscopic image viewer also includes a first movable viewing assembly and a second movable viewing assembly. The first and second movable viewing assemblies are contained within the housing.

The first movable viewing assembly includes the first lens and a first reflective structure. The first reflective structure is configured to reflect first visible light and to pass light reflected from a first eye of a user. The first reflective structure is positioned to reflect the first visible light into the first lens. The second movable viewing assembly includes the second lens and a second reflective structure. The second reflective structure is configured to reflect second visible light and to pass light reflected from a second eye of the user. The second reflective structure is positioned to reflect the second visible light into the second lens. In one aspect, the first reflective structure includes a dichroic surface, and the second reflective structure includes a dichroic surface. In another aspect, the first reflective structure includes a ninety percent reflective surface, and the second reflective structure includes a ninety percent reflective surface

In one aspect, the first lens is fixedly coupled to the first reflective structure in the first movable viewing assembly. The first lens and the first reflective structure are moved as a unit as the first movable viewing assembly moves. Similarly, the second lens is fixedly coupled to second reflective structure in the second movable viewing assembly. The second lens and the second reflective structure move as a unit as the second movable viewing assembly moves.

In yet another aspect, the first movable viewing assembly also includes a first display unit mounted in the first movable viewing assembly to provide the first visible light to the first reflective structure. The first display unit is fixedly coupled to the first reflective structure in the first movable viewing assembly. The first lens, the first reflective structure, and the first display unit move as a unit as the first movable viewing assembly moves. In this aspect, the second movable viewing assembly also includes a second display unit mounted in the second movable viewing assembly to provide the second visible light to the second reflective structure. The second display unit is fixedly coupled to the second reflective structure in the second movable viewing assembly. The second lens, the second reflective structure, and the second display unit move as a unit as the second movable viewing assembly moves.

In this apparatus the stereoscopic image viewer still further includes a viewer optic spacing adjustment assembly coupled to the first movable viewing assembly and to the second movable viewing assembly. The viewer optic spacing adjustment assembly is contained within the housing.

The apparatus also includes a controller coupled to the eye-tracking image capture unit and to the viewer optic spacing adjustment assembly. The controller is configured to measure an interpupillary distance using right and left eye images captured by the eye-tracking image capture unit from light reflected from a user's eyes, and is configured to command the viewer optic spacing adjustment assembly to adjust a distance between the first and second lenses to correspond to the measured interpupillary distance. The left and right eye images are included in a single frame captured by the eye-tracking image capture unit. The controller is also configured to recreate a known state of the stereoscopic image viewer using images of internal visible markings captured by the eye-tracking image capture unit.

In one aspect of this apparatus, the stereoscopic image viewer includes an illumination source contained within the housing. The illumination source is configured to provide non-visible light.

A stereoscopic image viewer includes a housing, a first lens, a second lens, and an eye-tracking image capture unit. The first lens, the second lens, and the eye-tracking image capture unit are mounted within the housing. The eye-tracking image capture unit has a field of view, and the eye-tracking image capture unit is mounted with the first lens and the second lens being within the field of view.

The stereoscopic image viewer also includes a first movable viewing assembly and a second movable viewing assembly. The first and second movable viewing assemblies are contained within the housing.

The first movable viewing assembly includes the first lens and a first reflective structure. The first reflective structure is configured to reflect first visible light and to pass light reflected from a first eye of a user. The first reflective structure is positioned to reflect the first visible light into the first lens.

The second movable viewing assembly includes the second lens and a second reflective structure. The second reflective structure being configured to reflect second visible light and to pass light reflected from a second eye of the user. The second reflective structure being positioned to reflect the second visible light into the second lens.

The first lens of the stereoscopic image viewer is fixedly coupled to the first reflective structure in the first movable viewing assembly. The first lens and the first reflective structure are moved as a unit as the first movable viewing assembly moves. Similarly, the second lens is fixedly coupled to second reflective structure in the second movable viewing assembly. The second lens and the second reflective structure move as a unit as the second movable viewing assembly moves.

In one aspect, the first movable viewing assembly of the stereoscopic image viewer also includes a first display unit mounted in the first movable viewing assembly to provide the first light to the first reflective structure. The first display unit is fixedly coupled to the first reflective structure in the first movable viewing assembly. The first lens, the first reflective structure, and the first display unit move as a unit as the first movable viewing assembly moves. Also, the second movable viewing assembly includes a second display unit mounted in the second movable viewing assembly to provide the second light to the second reflective structure. The second display unit is fixedly coupled to the second reflective structure in the second movable viewing assembly. The second lens, the second reflective structure, and the second display unit move as a unit as the second movable viewing assembly moves.

The stereoscopic image viewer still further includes a viewer optic spacing adjustment assembly coupled to the first movable viewing assembly and to the second movable viewing assembly. The viewer optic spacing adjustment assembly is contained within the housing.

In one aspect, the stereoscopic image viewer also includes an illumination source contained within the housing. The illumination source is configured, in one aspect, to provide non-visible light.

A method includes capturing, by an eye-tracking image capture unit contained within a stereoscopic image viewer, (i) a right eye image from first light reflected from a right eye of a user, the first reflected light passing through a first lens of the stereoscopic image viewer and a first reflective structure of the stereoscopic image viewer before being captured by the eye-tracking image capture unit and (ii) a left eye image from second light reflected from a left eye of the user, the second reflected light passing through a second lens of the stereoscopic image viewer and a second reflective structure of the stereoscopic image viewer before being captured by the eye-tracking image capture unit.

The method also includes determining, by a controller coupled to the eye-tracking image capture unit, an interpupillary distance of the user using the captured right eye image and the captured left eye image.

The method further includes commanding, by the controller, movement of the first lens and the second lens to positions having a separation corresponding to the interpupillary distance. In addition, in one aspect, the controller determines a distance between a first internal visible marker image captured with a captured right eye image and a second internal visible marker image captured with a left eye image and then commands movement of a first internal visible marker and a second internal visible marker to positions having a separation corresponding to a saved viewer optic spacing on the condition that the distance between the first internal visible marker image captured with the captured right eye image and the second internal visible marker image captured with the captured left eye image is not equal to the saved viewer optic spacing.

### BRIEF DESCRIPTION OF THE DRAWINGS

FIG. 1 is an illustration of a surgeon's console from a prior art teleoperated computer-assisted medical system.

FIG. 2 is an illustration of an apparatus that includes a stereoscopic image viewer and a controller.

FIG. 3 is a representation of information in a frame captured by an eye-tracking image capture unit of the stereoscopic image viewer of FIG. 2.

FIG. 4 is a schematic diagram of the stereoscopic image viewer of FIG. 2 with a schematic representation of a viewer optic spacing adjustment assembly that includes a rack-and-pinion gearset.

FIG. 5 is a high level process flow diagram for a method used by the controller of FIG. 2.

FIG. 6 is a more detailed block diagram of one aspect of the controller of FIG. 2.

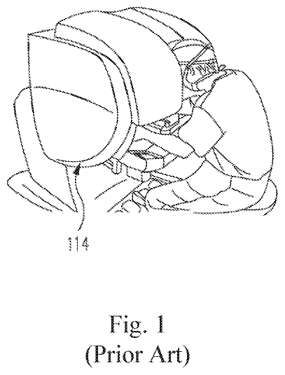

In the drawings, for single digit figure numbers, the first digit in the reference numeral of an element is the number of the figure in which that element first appears.

### DETAILED DESCRIPTION

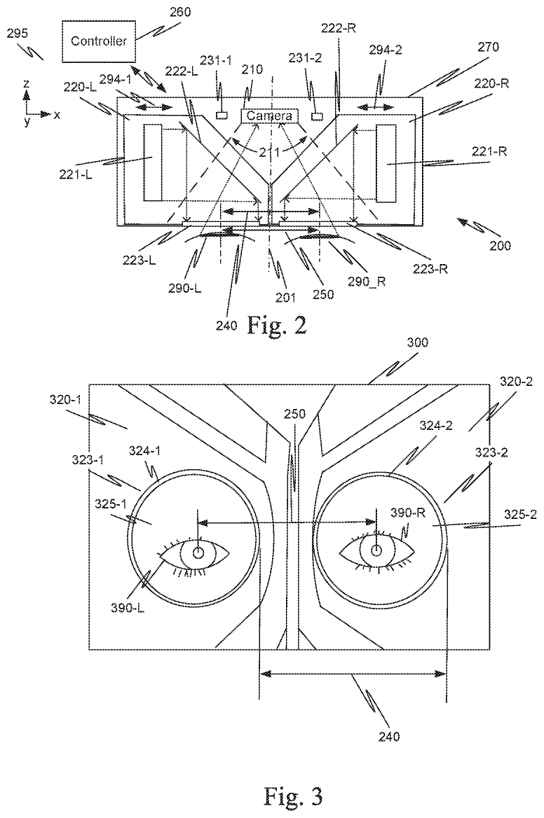

A stereoscopic image viewer 200 (FIG. 2) includes a single eye-tracking image capture unit 210, e.g., a camera, mounted within stereoscopic image viewer 200. In one aspect, eye-tracking image capture unit 210 captures a frame that includes an image of each of a user's eyes 290-L, 290-R. A controller 260 coupled to stereoscopic image viewer 200 receives the captured frame, and determines an interpupillary distance 250 between the user's eyes using information from the captured frame, i.e., using image data captured by eye-tracking image capture unit 210. If a viewer optic spacing 240 is not equal to interpupillary distance 250, controller 260 commands stereoscopic image viewer 200 to change viewer optic spacing 240 to match interpupillary distance 250.

In another aspect, controller 260 recreates a known state between some internal visible markings, so that stereoscopic image viewer 200 has interpupillary distance 250 for the current user. For example, if there is a saved viewer optic spacing SVOS for a user, when that user logs in to use stereoscopic image viewer 200, controller 260 retrieves the saved user viewer optic spacing SVOS, and then commands stereoscopic image viewer 200 to move components of stereoscopic image viewer 200 to recreate the known state between the internal visible markings that corresponds to saved viewer optic spacing SVOS. (Here, internal means internal to stereoscopic image viewer 200.) Specifically, controller 260 configures stereoscopic image viewer 200 so that the internal visible markings, e.g., a first internal visible marking and a second internal visible marking, are separated by a distance equal to saved viewer optic spacing SVOS. Thus, in this aspect, it is unnecessary to determine a user's interpupillary distance 250 every time the user accesses stereoscopic image viewer 200, because controller 260 uses saved user viewer optic spacing SVOS to automatically configure stereoscopic image viewer 200 to match the user's interpupillary distance 250.

Herein, a single controller 260 is referenced and described. Although described as a single controller, it is to be appreciated that this controller may be implemented in practice by any one of or any combination of hardware, software that is executed on a processor, and firmware. Also, a controller's functions, as described herein, may be performed by one unit or divided up among different components, each of which may be implemented in turn by any one of or any combination of hardware, software that is executed on a processor, and firmware. When divided up among different components, the components may be centralized in one location or distributed across a computer-aided medical system for distributed processing purposes.

A processor should be understood to include at least a logic unit and a memory associated with the logic unit. Thus, in various embodiments, controller 260 includes programmed instructions (e.g., a non-transitory machine-readable medium storing the instructions) to implement some or all of the methods described in accordance with aspects disclosed herein. Any of a wide variety of centralized or distributed data processing architectures may be employed. Similarly, the programmed instructions may be implemented as a number of separate programs or subroutines, or they may be integrated into a number of other aspects of the systems described herein.

Controller 260 can be coupled to stereoscopic image viewer 200 by a wired connection, a wireless connection, an optical connection, etc. In some embodiments, controller 260 supports wireless communication protocols such as Bluetooth, Infrared Data Association (IrDA) protocol, Home Radio Frequency (HomeRF) protocol, IEEE 802.11 protocol, Digital Enhanced Cordless Telecommunications (DECT) protocol, and Wireless Telemetry protocol.

Use of a single eye-tracking image capture unit 210 to simultaneously capture images of both eyes of a user in a single frame overcomes limitations of prior art techniques that utilized two cameras. The single eye-tracking image capture unit eliminates problems associated with spatial register and/or temporal registration associated with images captured by two different cameras. Also, use of a single camera eliminates hardware and controller complexity associated with two cameras. Placement of eye-tracking image capture unit 210 between the viewing optics, as opposed to in front of the viewing optics, also minimizes problems associated with user head movement and the number of cameras required to reliably capture the required data for eye-tracking.

Eye-tracking image capture unit 210 is mounted within a housing 270 of stereoscopic image viewer 200, i.e., is contained within housing 270. The viewing optics of stereoscopic image viewer 200, i.e., lens assemblies 223-L, 223-R, are mounted in stereoscopic image viewer 200 so that the viewing optics are between a user of stereoscopic image viewer 200 and a lens assembly of eye-tracking image capture unit 210. Thus, with reference to eyes 290-L, 290-R of a user of stereoscopic image viewer 200, eye-tracking image capture unit 210 is mounted behind the viewing optics.

For ease of discussion, a Cartesian x-y-z coordinate system 295 is defined with respect to stereoscopic image viewer 200. Note, FIG. 2 is a top view of stereoscopic image viewer 200. In this top view, a z-axis (a third axis) extends in the up and down directions in FIG. 2. A y-axis (a second axis) extends into and out of the page while a x-axis (a first axis) extends in the left and right directions (first and second directions).

The location of eye-tracking image capture unit 210 relative to the viewing optics within housing 270 is not critical so long as a field of view 211 of eye-tracking image capture unit 210 includes first lens assembly 223-L and second lens assembly 223-R. This assures that first light reflected from eye 290-L (represented by dotted line between eye 290-L and eye-tracking image capture unit 210) and second light reflected from eye 290-R (represented by dotted line between eye 290-R and eye-tracking image capture unit 210) can be captured in a single frame by eye-tracking image capture unit 210. In this example, a lens of eye-tracking image capture unit 210 is centered on an axis 201 that extends in the z-direction of stereoscopic image viewer 200.

In addition to eye-tracking image capture unit 210, stereoscopic image viewer 200 also includes a first movable viewing assembly 220-L and a second movable viewing assembly 220-R. In this aspect, second movable viewing assembly 220-R is a mirror image of first movable viewing assembly 220-L about axis 201.

First movable viewing assembly 220-L includes a first display device 221-L, a first reflective structure 222-L, and a first lens assembly 223-L. First display device 221-L, first reflective structure 222-L, and first lens assembly 223-L are mounted in a fixed spatial relationship to each other in first viewing assembly 220-L. Thus, translating first movable viewing assembly 220-L in first and second directions 294-1 along the x-axis moves not only first lens assembly 223-L in the first and second directions 294-1, but also first display device 221-L and first reflective structure 222-L. Consequently, the fixed spatial relationship between first display device 221-L, first reflective structure 222-L and first lens assembly 223-L assures that a first visible light image output by first display device 221-L is reflected by first reflective structure 222-L to first lens assembly 223-L irrespective of the position of first lens assembly 223-L along the x-axis.

Second movable viewing assembly 220-R includes a second display device 221-R, a second reflective structure 222-R, and a second lens assembly 223-R. Second display device 221-R, second reflective structure 222-R, and second lens assembly 223-R are mounted in a fixed spatial relationship to each other in second viewing assembly 220-R. Thus, translating second viewing assembly 220-R in first and second directions 294-2 along the x-axis moves not only second lens assembly 223-R in the first and second directions 294-2, but also second display device 221-R and second reflective structure 222-R. Consequently, the fixed spatial relationship between second display device 221-R, second reflective structure 222-R and second lens assembly 223-R assures that a second visible light image output by second display device 221-R is reflected by second reflective structure 222-R to second lens assembly 223-R irrespective of the position of second lens assembly 223-R along the x-axis.

In one aspect, the first visible light image output from first display device 221-L and the second visible light image output from second display device 221-R are left and right scenes, respectively, captured by an endoscope or other medical instrument when stereoscopic image viewer 200 is part of a computer-assisted medical system, e.g., mounted in surgeon's console 114. Typically, the first and second visible light images are color images, but also could be monochrome, black and white, or gray-scale images.

In one aspect, each of first and second lens assemblies 223-L and 223-R includes a viewing lens and a lens retainer ring. (See FIG. 3.) The lens retainer ring holds the viewing lens in the viewing assembly. In one aspect of the viewing lenses, each viewing lens is made up of plurality of individual lens, e.g., three lenses, four lenses, etc., that are glued together to form the viewing lens.

The characteristics of reflective structures 222-L and 222-R are selected based on the characteristics of the reflected light from an eye of a user, which is captured by eye-tracking image capture unit 210. If the reflected light is visible light, reflective structures 222-L and 222-R are, for example, one-way mirrors. If the reflected light is non-visible light, e.g., infrared light, reflective structures 222-L and 222-R are dichroic structures that reflect some visible light and pass non-visible light. In yet another aspect, each of reflective structures 222-L and 222-R is a ninety percent reflective mirror on black glass. A ninety percent reflective mirror is a surface that reflects ninety percent of the incident light and passes ten percent of the incident light. Thus, in this aspect, image capture unit 210 sees ten percent of the light reflected from first and second lens assemblies 223-L and 223-R and ten percent of the light reflected from a user's eyes.

Stereoscopic image viewer 200 optionally includes an illuminator that is used if other than visible light is captured by eye-tracking image capture unit 210. If non-visible light is captured by eye-tracking image capture unit 210, the optional illuminator is used, and the optional illuminator includes a plurality of non-visible illumination sources 231-1 and 231-2 that are mounted adjacent to eye-tracking image capture unit 210. In one aspect, the non-visible illumination sources are infrared illumination sources. If non-visible illumination sources 231-1 and 231-2 are used in stereoscopic image viewer 200, a visible light filter is used on eye-tracking image capture unit 210 to block capture of visible light. For example if non-visible illumination sources are 880 nm light emitting diodes, the filter passes light above 830 nm into eye-tracking image capture unit 210.

FIG. 3 is a representation of a scene in a frame 300 captured by eye-tracking image capture unit 210. The scene includes an image 320-1 of a portion of first movable viewing assembly 220-L and an image 320-2 of a portion of second movable viewing assembly 220-R. Image 320-1 includes an image 323-1 of first lens assembly 223-L. Image 323-1 includes an image 324-1 of a first lens retainer ring and an image 325-1 of a first lens. Similarly, image 320-2 includes an image 323-2 of second lens assembly 223-R. Image 323-2 includes an image 324-2 of a second lens retainer ring and an image 325-2 of a second lens.

In one aspect, the components of stereoscopic image viewer 200 shown in frame 300, with the exception of the lens retainer rings and the lenses in this example, are coated with an anti-reflective coating to minimize stray reflected light that is captured by eye-tracking image capture unit 210. In another aspect, the outer perimeter of each viewing lens is coated with an anti-reflective coating, and in still yet another aspect, the outer perimeter of each lens making up the viewing lens is coated with an anti-reflective coating.

In the aspect, discussed below, the images of the lens rings are used as internal visible markings in determining the viewer optic spacing. However, this is illustrative only and is not intended to be limiting. A common feature on each of first movable viewing assembly 220-L and second movable viewing assembly 220-R could be the internal visible marking used in determining the spacing between first movable viewing assembly 220-L and second movable viewing assembly 220-R, and consequently the spacing between the lenses in the two assemblies. This common feature could be an internal physical structure or an internal marker, e.g., an indentation, a tab, a painted symbol, etc. that is visible in the captured image of the user's eyes. If images of internal features other the images of the lens rings are used as the visible markings in determining the viewer optic spacing, the internal features are not coated with the anti-reflective coating and the lens rings are coated with the anti-reflective coating.

An image 390-L of the user's left eye 290-L and an image 390-R of the user's right eye 290L are also captured in frame 300. Each image of the eye includes an image of the iris and an image of the pupil. Other features of the user's face in the vicinity of the eyes are typically also captured in frame 300, but these are not shown in FIG. 3, because the other features are not needed to understand the novel aspects described herein. Frame 300 also includes an image of a first internal visible marking and an image of a second internal visible marking. In frame 300, the images of the lens rings are used as examples of an image of a first internal visible marking and an image of a second internal visible marking that are captured with the images of the user's eyes.

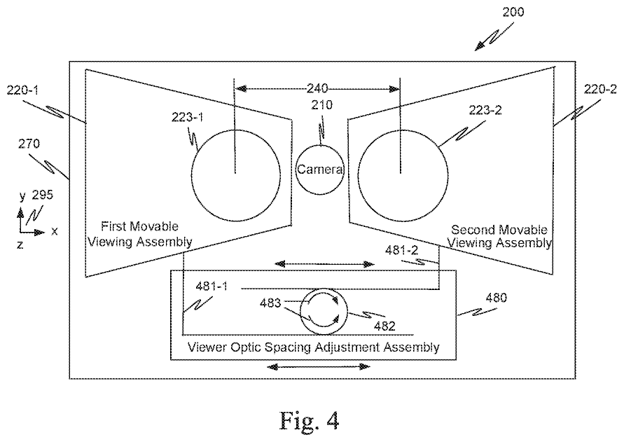

FIG. 4 is a schematic diagram of stereoscopic image viewer 200 that includes a schematic representation of a viewer optic spacing adjustment assembly 480 that includes, in this example, a rack-and-pinion gear set. Specifically, a first rack 481-1 is connected to first movable viewing assembly 220-L and is engaged with, i.e., coupled to, pinion 482. A second rack 481-2 is connected to second movable viewing assembly 220-R and is engaged with, i.e., coupled to, pinion 482. Pinion 482 is driven by an actuator, e.g., an electric motor. Controller 260 commands the actuator to turn pinion 482, which in turn moves racks 481-1 and 481-2, and consequently first and second movable viewing assemblies 220-L and 220-R.

As explained below, controller 260 commands the actuator to turn pinion 482 until a spacing between the visible markings (as determined from the captured images) equals a saved viewer optic spacing or until a spacing between the visible markings, i.e., the viewer optic spacing, is equal to the interpupillary distance of the user.

In one aspect, viewer optic spacing 240 can be varied by viewer optic spacing adjustment assembly 480 from 54 mm to 74 mm. Thus, controller 260 can vary the positioning of first and second movable viewing assemblies 220-L and 220-R along an X-axis to accommodate an interpupillary distance 250 of between 54 mm and 74 mm. As used herein, interpupillary distance 250 is the distance between the centers of the pupils of the eyes. It has been reported that an average interpupillary distance is about 62 mm for women and about 64 mm for men.

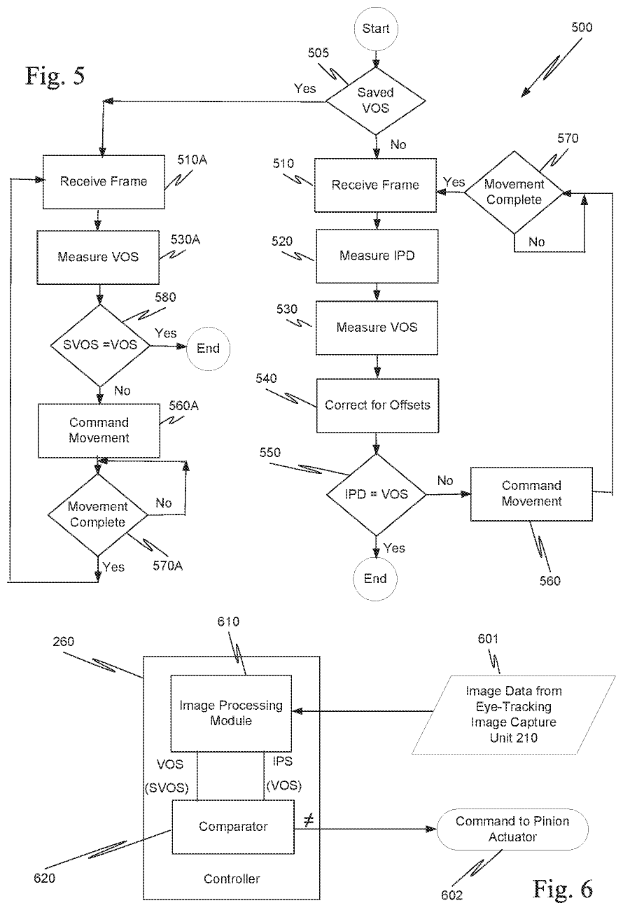

FIG. 5 is a high level process flow diagram for a method 500 used by controller 260 to match viewer optic spacing 240 with interpupillary distance 250 of a user. In a first branch, a saved viewer optic spacing SVOS is not available, and so controller 260 finds a viewer optic spacing 240 that matches the user's interpupillary distance 250. In a second branch, a saved viewer optic spacing SVOS is available, and so controller 260 sets viewer optic spacing 240 to be equal to saved viewer optic spacing SVOS, e.g., configures stereoscopic image viewer 200 to have a known viewer optic spacing.

The use of two linear branches in method 500 is illustrative only and is not intended to be limiting. In view of FIG. 5, one of skill in the art can implement the method in a variety of ways—depending on whether a saved viewer optic spacing is available for the current user—so that viewer optic spacing 240 is equal to the current user's interpupillary distance 250.

Prior to considering method 500 in further detail, it is noted that feature extraction from information in a captured frame is known to those knowledgeable in the field, and so is not considered in detail herein. Further, techniques for detecting the pupil and/or iris in an image of eye using feature extraction are also known, and so are not considered in detail herein. Similarly, determining the distance between pupils and determining distances between features extracted from a captured frame, e.g., a distance between images of internal visible markings, is also known and so is considered in detail herein.

Initially, SAVED VIEWER OPTIC SPACING (SAVED VOS) check process 505 determines, for a current user, whether a saved viewer optic spacing SVOS is available. In one aspect, a saved viewer optic spacing SVOS is saved with user login and configuration data for a teleoperated surgical system. If a saved viewer optic spacing SVOS is unavailable, SAVED VIEWER OPTIC SPACING (SAVED VOS) check process 505 transfers processing to RECEIVE FRAME act 510 in the first branch of method 500. Conversely, if a saved viewer optic spacing SVOS is available, SAVED VIEWER OPTIC SPACING (SAVED VOS) check process 505 transfers processing to RECEIVE FRAME act 510A in the second branch of method 500.

In RECEIVE FRAME act 510, a frame captured by eye-tracking image capture unit 210 is received. Upon receipt of the captured frame, processing transfers from RECEIVE FRAME act 510 to MEASURE INTERPUPILLARY DISTANCE (IPD) act 520.

MEASURE INTERPUPILLARY DISTANCE act 520 first locates the two pupils in the captured frame. After the two pupils are located, an interpupillary distance 250 between the centers of the pupils is determined. In one aspect, this interpupillary distance 250 is specified in pixels. Upon completion of MEASURE INTERPUPILLARY DISTANCE act 520, processing transfers from MEASURE INTERPUPILLARY DISTANCE act 520 to MEASURE VIEWER OPTIC SPACING (VOS) act 530.

In MEASURE VIEWER OPTIC SPACING act 530, the distance between the centers of the lens of stereoscopic image viewer 200 is measured. This can be done in a number of ways. For example, a first internal visible marker can be placed on first movable viewing assembly 220-L and a second internal visible marker can be placed on second movable viewing assembly 220-R. The first and second internal visible markers are within field of view 211 of eye-tracking image capture unit 210 and have a known geometrical relationship with eye-tracking image capture unit 210. The distance between the first and second internal visible markers corresponds to viewer optic spacing 240. Thus, in this aspect, images of the first and second internal visible markers would be extracted from the received frame and the distance between the markers would be measured to determine viewer optic spacing 240. In one aspect, the first and second internal visible markers are on first lens assembly 223-L and second lens assembly 223-R, respectively.

In another aspect, for the captured frame illustrated in FIG. 3, each of the lenses and its retaining ring is circular, and so a distance between lines tangent at the same corresponding point on each of the retaining rings along the x-axis is viewer optic spacing 240. Hence, for this example, MEASURE VIEWER OPTIC SPACING act 530 extracts image 324-1 of the first lens retainer ring and image 324-2 of second retainer ring. A distance between a first point on the outer circumference of image 324-1 of the first retainer ring at the ninety degree (three o'clock) location and a second point on the outer circumference of image 324-1 of the second retainer ring at the ninety degree (three o'clock) location is measured. This distance is viewer optic spacing 240. In one aspect, viewer optic spacing 240 is measured in pixels. Note that even if each of the lens and its lens retainer ring are not circular, this technique still can be used so long as the point chosen on the circumference of the retainer ring has a fixed known relationship to the center of the lens. Also, the use of pixels to measure distances is illustrative only and is not intended to be limiting. In view of this disclosure, any normalized system of measurement could be used to measure the distances of interest. Upon completion of MEASURE VIEWER OPTIC SPACING act 530, processing transfers from MEASURE VIEWER OPTIC SPACING act 530 to CORRECT FOR OFFSETS act 540.

INTERPUPILLARY DISTANCE EQUALS VIEWER OPTIC SPACING check process 550 determines whether interpupillary distance 250 is equal to viewer optic spacing 240. However the interpupillary distance determined in MEASURE INTERPUPILLARY DISTANCE act 520 cannot be directly compared with the viewer optic spacing determined in MEASURE VIEWER OPTIC SPACING act 530 because the lenses and the pupils are at different distances from eye-tracking image capture unit 210. Thus, in CORRECT FOR OFFSETS act 540, the two measurements are transformed using the known geometry so that a direct comparison of interpupillary distance 250 with viewer optic spacing 240 can be made.

In one aspect in a computer-assisted medical system, stereoscopic image viewer 200 includes a presence sensor which indicates whether the user is in contact with stereoscopic image viewer 200. If this presence sensor indicates the user is in contract with stereoscopic image viewer, the geometric relationship between the user's eyes 290-L and 290-R, lens assemblies 223-L and 223-R, and eye-tracking image capture unit 210 is known. Thus, CORRECT FOR OFFSETS act 540 uses the known geometrical relationship to compensate for the differing offsets from eye-tracking image capture unit 210. In another aspect, even if the presence sensor does not indicate the user's presence, CORRECT FOR OFFSETS act 540 uses the known geometric relationship between the user's eyes 290-L and 290-R when the user's head is in contact with stereoscopic image viewer 200, lens assemblies 223-L an 223-R, and eye-tracking image capture unit 210 to compensate for the different offsets between the user's eyes 290-L and 290-R and lens assemblies 223-L an 223-R from eye-tracking image capture unit 210.

In still yet another aspect, prior to using stereoscopic image viewer 200 in a clinical setting, a user is positioned at a plurality of distances from stereoscopic image viewer 200 as well as in contact with stereoscopic image viewer 200, and a frame is captured by eye-tracking image capture unit 210 at each of the positions. Each of these images is analyzed and the size of the user's irises at each position is saved along with the position in the user's login and configuration data for the computer-assisted medical system. In this aspect, if the presence sensor in stereoscopic image viewer 200 does not indicate the presence of the user, CORRECT FOR OFFSETS act 540 heuristically determines the offset of eyes 290-L and 290-R from stereoscopic image viewer 200 using the size of the user's irises in frame 300 and the stored iris size vs. distance data. For example, an interpolation of the distance is obtained using the stored iris sizes by assuming that the variation in both iris size and distance is linear. With the interpolated offset from stereoscopic image viewer 200 and the known geometry of stereoscopic image viewer 200, CORRECT FOR OFFSETS act 540 compensates for the different offsets between the user's eyes 290-L and 290-R and lens assemblies 223-L an 223-R from eye-tracking image capture unit 210 so that interpupillary distance 250 can be directly compared to viewer optic spacing 240.

The above examples of correcting the offset when a user is not completely in stereoscopic image viewer 200 are illustrative only and are not intended to be limiting. Other heuristic approaches could be used. For example, a group of people could be used to generate data of iris size vs distance from stereoscopic image viewer 200. This data could be analyzed and used to generate a number that is used to adjust the user eye offset based on iris size in FIG. 3, for example. In this aspect, the offset correction would not be user specific.

Upon completion of CORRECT FOR OFFSETS act 540, interpupillary distance 250 and viewer optic spacing 240 are corrected for the differing offsets such that interpupillary distance 250 and viewer optic spacing 240 can be directly compared. Thus, processing transfers from CORRECT FOR OFFSETS act 540 to INTERPUPILLARY DISTANCE EQUALS VIEWER OPTIC SPACING check process 550.

INTERPUPILLARY DISTANCE EQUALS VIEWER OPTIC SPACING check process 550 compares interpupillary distance 250 and viewer optic spacing 240. If interpupillary distance 250 is equal to viewer optic spacing 240, no action is necessary and so method 500 stops. However, in one aspect, prior to stopping, controller 260 saves viewer optic spacing 240 in the login and configuration data for the current user. If interpupillary distance 250 is not equal to viewer optic spacing 240, processing transfers from INTERPUPILLARY DISTANCE EQUALS VIEWER OPTIC SPACING check process 550 to COMMAND MOVEMENT act 560.

In one aspect, processing waits in COMMAND MOVEMENT act 560 until a user input is received to adjust viewer optic spacing 240, and then a command is sent to viewer optic spacing adjustment assembly 480 to adjust viewer optic spacing 240. In another aspect, COMMAND MOVEMENT act 560 automatically sends a command to viewer optic spacing adjustment assembly 480 to adjust viewer optic spacing 240. Upon completion of COMMAND MOVEMENT ACT 560, processing transfers to MOVEMENT COMPLETE check process 570.

Processing remains in MOVEMENT COMPLETE check process 570, until viewer optic spacing adjustment assembly 480 completes the commanded change to adjust viewer optic spacing 240 and then transfers to RECEIVE FRAME act 510. MOVEMENT COMPLETE check process 570 should not be interpreted as requiring continuous polling to determine when viewer optic spacing adjustment assembly 480 completes the commanded change to adjust viewer optic spacing 240. Rather, processing is delayed after COMMAND MOVEMENT act 560 until viewer optic spacing adjustment assembly 480 has time to complete the commanded change to adjust viewer optic spacing 240, and then processing transfers to RECEIVE FRAME act 510, and at least acts 510 to 550 are repeated.

In one aspect, if the frame rate of captured images is high enough relative to the speed at which viewer optic spacing adjustment assembly 480 moves first movable viewing assembly 220-L and second movable viewing assembly 220-R, it is possible to command viewer optic spacing adjustment assembly 480 to move first movable viewing assembly 220-L and second movable viewing assembly 220-R continuously, and then stop when the interpupillary distance 250 equals viewer optic spacing 240. Here, “high enough” means that a plurality of frames are captured and analyzed, between each increment of distance that viewer optic spacing adjustment assembly 480 moves first movable viewing assembly 220-L and a second movable viewing assembly 220-R. In this aspect, MOVEMENT COMPLETE check process 570 and COMMAND MOVEMENT act 560 would not be needed after INTERPUPILLARY DISTANCE EQUALS VIEWER OPTIC SPACING check process 550. Instead, COMMAND MOVEMENT act 560 would be performed prior to the first instance of RECEIVE FRAME act 510, and INTERPUPILLARY DISTANCE EQUALS VIEWER OPTIC SPACING check process 550 would transfer directly to RECEIVE FRAME act 510 when interpupillary distance is not equal to viewer optic spacing 240.

As indicated above, if a saved viewer optic spacing SVOS is available, SAVED VIEWER OPTIC SPACING (SAVED VOS) check process 505 transfers processing to RECEIVE FRAME act 510A in the second branch of method 500.

In RECEIVE FRAME act 510A, a frame captured by eye-tracking image capture unit 210 is received. Upon receipt of the captured frame, processing transfers from RECEIVE FRAME act 510A to MEASURE VIEWER OPTIC SPACING act 530A.

In MEASURE VIEWER OPTIC SPACING act 530A, the distance between the centers of the lens of stereoscopic image viewer 200 is measured using images of internal visible markings that are captured with images of the user's eyes. MEASURE VIEWER OPTIC SPACING act 530A is the same as MEASURE VIEWER OPTIC SPACING act 530, and so that description is not repeated here. Upon completion of MEASURE VIEWER OPTIC SPACING act 530A, processing transfers from MEASURE VIEWER OPTIC SPACING act 530A to SAVED VIEWER OPTIC SPACING EQUALS VIEWER OPTIC SPACING check process 580.

SAVED VIEWER OPTIC SPACING EQUALS VIEWER OPTIC SPACING check process 580 compares saved viewer optic spacing SVOS and viewer optic spacing 240. If saved viewer optic spacing SVOS is equal to viewer optic spacing 240, no action is necessary and so method 500 stops. If saved viewer optic spacing SVOS is not equal to viewer optic spacing 240, processing transfers from SAVED VIEWER OPTIC SPACING EQUALS VIEWER OPTIC SPACING check process 580 transfers processing to COMMAND MOVEMENT act 560A. COMMAND MOVEMENT act 560A is equivalent to COMMAND MOVEMENT act 560.

COMMAND MOVEMENT act 560A automatically sends a command to viewer optic spacing adjustment assembly 480 to adjust viewer optic spacing 240. Upon completion of COMMAND MOVEMENT ACT 560, processing transfers to MOVEMENT COMPLETE check process 570A. MOVEMENT COMPLETE check process 570A is equivalent to MOVEMENT COMPLETE check process 570.

Processing remains in MOVEMENT COMPLETE check process 570A, until viewer optic spacing adjustment assembly 480 completes the commanded change to adjust viewer optic spacing 240 and then transfers to RECEIVE FRAME act 510A. When processing transfers to RECEIVE FRAME act 510, at least acts 510A to 580 are repeated.

In one aspect, if the frame rate of captured images is high enough relative to the speed at which viewer optic spacing adjustment assembly 480 moves first movable viewing assembly 220-L and second movable viewing assembly 220-R, it is possible to command viewer optic spacing adjustment assembly 480 to move first movable viewing assembly 220-L and second movable viewing assembly 220-R continuously, and then stop when saved viewer optic spacing SVOS equals viewer optic spacing 240. In this aspect, MOVEMENT COMPLETE check process 570A and COMMAND MOVEMENT act 560A would not be needed after SAVED VIEWER OPTIC SPACING EQUALS VIEWER OPTIC SPACING check process 580. Instead, COMMAND MOVEMENT act 560A would be performed prior to the first instance of RECEIVE FRAME act 510A, and SAVED VIEWER OPTIC SPACING EQUALS VIEWER OPTIC SPACING check process 580 would transfer directly to RECEIVE FRAME act 510A when saved viewer optic spacing SVOS is not equal to viewer optic spacing 240.

FIG. 6 is a more detailed block diagram of one aspect of controller 260. In this aspect, controller 260 includes an image processing module 610 and comparator logic 620. If there is not a saved viewer optic spacing for the current user, image processing module 610 receives a frame 601 of image data from eye-tracking image capture unit 210 in RECEIVE FRAME act 510 and then performs each of acts 520 to 540. Image processing module 610 transfers the offset corrected interpupillary distance IPD and viewer optic spacing VOS to comparator logic 620. If interpupillary distance IPD and viewer optic spacing VOS are not equal, controller 260 sends a command 602 to the pinion actuator in viewer optic spacing adjustment assembly 480.

If there is a saved viewer optic spacing for the current user, image processing module 610 receives a frame 601 of image data from eye-tracking image capture unit 210 in RECEIVE FRAME act 510A and then performs each of acts 510A and 530A. Image processing module 610 transfers the saved viewer optic spacing SVOS and viewer optic spacing VOS to comparator logic 620. If saved viewer optic spacing SVOS and viewer optic spacing VOS are not equal, controller 260 sends a command 602 to the pinion actuator in viewer optic spacing adjustment assembly 480.

As used herein, “first,” “second,” “third,” “fourth,” etc. are adjectives used to distinguish between different components or elements. Thus, “first,” “second,” “third,” “fourth,” etc. are not intended to imply any ordering of the components or elements.

The above description and the accompanying drawings that illustrate aspects and embodiments of the present inventions should not be taken as limiting—the claims define the protected inventions. Various mechanical, compositional, structural, electrical, and operational changes may be made without departing from the spirit and scope of this description and the claims. In some instances, well-known circuits, structures, and techniques have not been shown or described in detail to avoid obscuring the invention.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of the device in use or operation in addition to the position and orientation shown in the figures. For example, if the device in the figures is turned over, elements described as “below” or “beneath” other elements or features would then be “above” or “over” the other elements or features. Thus, the exemplary term “below” can encompass both positions and orientations of above and below. The device may be otherwise oriented (rotated 90 degrees or at other orientations) and the spatially relative descriptors used herein interpreted accordingly. Likewise, descriptions of movement along and around various axes include various special device positions and orientations.

The singular forms “a”, “an”, and “the” are intended to include the plural forms as well, unless the context indicates otherwise. The terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

As explained above, the controller described herein can be implemented by software executing on a processor, hardware, firmware, or any combination of the three. When the controller is implemented as software executing on a processor, the software is stored in a memory as computer readable instructions and the computer readable instructions are executed on the processor. All or part of the memory can be in a different physical location than a processor so long as the processor can be coupled to the memory. Memory refers to a volatile memory, a non-volatile memory, or any combination of the two.

Also, the functions of the controller, as described herein, may be performed by one unit, or divided up among different components, each of which may be implemented in turn by any combination of hardware, software that is executed on a processor, and firmware. When divided up among different components, the components may be centralized in one location or distributed across the system for distributed processing purposes. The execution of the controller results in methods that perform the processes described above for the controller.

A processor is coupled to a memory containing instructions executed by the processor. This could be accomplished within a computer system, or alternatively via a connection to another computer via modems and analog lines, or digital interfaces and a digital carrier line, or via connections using any of the protocols described above. In view of this disclosure, instructions used in any part of or all of the processes described herein can be implemented in a wide variety of computer system configurations using an operating system and computer programming language of interest to the user.

Herein, a computer program product comprises a computer readable medium configured to store computer readable code needed for any part of or all of the processes described herein, or in which computer readable code for any part of or all of those processes is stored. Some examples of computer program products are CD-ROM discs, DVD discs, flash memory, ROM cards, floppy discs, magnetic tapes, computer hard drives, servers on a network and signals transmitted over a network representing computer readable program code. A non-transitory tangible computer program product comprises a tangible computer readable medium configured to store computer readable instructions for any part of or all of the processes or in which computer readable instructions for any part of or all of the processes is stored. Non-transitory tangible computer program products are CD-ROM discs, DVD discs, flash memory, ROM cards, floppy discs, magnetic tapes, computer hard drives, and other physical storage mediums.

All examples and illustrative references are non-limiting and should not be used to limit the claims to specific implementations and embodiments described herein and their equivalents. Any headings are solely for formatting and should not be used to limit the subject matter in any way, because text under one heading may cross reference or apply to text under one or more headings. Finally, in view of this disclosure, particular features described in relation to one aspect or embodiment may be applied to other disclosed aspects or embodiments of the invention, even though not specifically shown in the drawings or described in the text.

1. An apparatus comprising:
a stereoscopic image viewer comprising:
a housing, a first lens, a second lens, and an eye-tracking image capture unit;
the first lens, the second lens, and the eye-tracking image capture unit being contained in the housing; and
the eye-tracking image capture unit having a field of view and the eye-tracking image capture unit being mounted within the housing with the first lens and the second lens within the field of view.

2. The apparatus of claim 1, the stereoscopic image viewer further comprising:
a first movable viewing assembly comprising the first lens and a first reflective structure, the first reflective structure reflecting first visible light and passing light reflected from a first eye of a user, and the first reflective structure being positioned to reflect the first visible light into the first lens; and
a second movable viewing assembly comprising the second lens and a second reflective structure, the second reflective structure reflecting a second visible light and passing light reflected from a second eye of the user, and the second reflective structure being positioned to reflect the second visible light into the second lens, and the first and second movable viewing assemblies being contained within the housing.

3. The apparatus of claim 2, wherein the first reflective structure comprises a dichroic surface, and wherein the second reflective structure comprises a dichroic surface.

4. The apparatus of claim 2, wherein the first reflective structure comprises a ninety percent reflective surface, and wherein the second reflective structure comprises a ninety percent reflective surface.

5. The apparatus of claim 2:
the first lens being fixedly coupled to the first reflective structure in the first movable viewing assembly, wherein the first lens and the first reflective structure are moved as a unit as the first movable viewing assembly moves; and
the second lens being fixedly coupled to second reflective structure in the second movable viewing assembly, wherein the second lens and the second reflective structure move as a unit as the second movable viewing assembly moves.

6. The apparatus of claim 5:
the first movable viewing assembly further comprising a first display unit mounted in the first movable viewing assembly to provide the first visible light to the first reflective structure, the first display unit being fixedly coupled to the first reflective structure in the first movable viewing assembly, wherein the first lens, the first reflective structure, and the first display unit move as a unit as the first movable viewing assembly moves; and
the second movable viewing assembly further comprising a second display unit mounted in the second movable viewing assembly to provide the second visible light to the second reflective structure, the second display unit being fixedly coupled to the second reflective structure in the second movable viewing assembly, wherein the second lens, the second reflective structure, and the second display unit move as a unit as the second movable viewing assembly moves.

7. The apparatus of claim 2, the stereoscopic image viewer further comprising:
a viewer optic spacing adjustment assembly coupled to the first movable viewing assembly and to the second movable viewing assembly, the viewer optic spacing adjustment assembly being contained within the housing.

8. The apparatus of claim 7, further comprising:
a controller coupled to the eye-tracking image capture unit and to the viewer optic spacing adjustment assembly, the controller being configured to:
measure an interpupillary distance using right and left eye images captured by the eye-tracking image capture unit from light reflected from a user's eyes; and
command the viewer optic spacing adjustment assembly to adjust a distance between the first and second lenses to correspond to the measured interpupillary distance.

9. The apparatus of claim 1, the stereoscopic image viewer further comprising:
an illumination source contained within the housing, the illumination source being configured to provide non-visible light.

10. The apparatus of claim 1, further comprising:
a controller coupled to the eye-tracking image capture unit, the controller being configured to measure an interpupillary distance using left and right eye images captured by the eye-tracking image capture unit from light reflected from a user's eyes.

11. The apparatus of claim 10, the controller further being configured to command adjustment of a spacing between the first lens and the second lens to correspond to the measured interpupillary distance.

12. The apparatus of claim 10, the controller further being configured to command a viewer optic spacing adjustment assembly coupled to the first lens and the second lens to adjust a spacing between the first lens and the second lens to correspond to the measured interpupillary distance.

13. The apparatus of claim 10, wherein the left and right eye images are included in a single frame captured by the eye-tracking image capture unit.

14. The apparatus of claim 13, further comprising: the controller further being configured to determine a current spacing between the first lens and the second lens from information captured in the single frame.

15. The apparatus of claim 14, the controller further being configured to command a viewer optic spacing adjustment assembly coupled to the first lens and the second lens to adjust a spacing between the first lens and the second lens to correspond to the measured interpupillary distance.

16. The apparatus of claim 1, further comprising:
a controller coupled to the eye-tracking image capture unit, the controller being configured to recreate a known state of the stereoscopic image viewer using images of internal visible markings captured by the eye-tracking image capture unit.

17. The apparatus of claim 1, wherein the first and second lenses are completely within the field of view of the eye-tracking image capture unit.

18-24. (canceled)

25. A method comprising:
capturing, by an eye-tracking image capture unit contained within a stereoscopic image viewer, (i) a right eye image from first light reflected from a right eye of a user, the first reflected light passing through a first lens of the stereoscopic image viewer and a first reflective structure of the stereoscopic image viewer before being captured by the eye-tracking image capture unit and (ii) a left eye image from second light reflected from a left eye of the user, the second reflected light passing through a second lens of the stereoscopic image viewer and a second reflective structure of the stereoscopic image viewer before being captured by the eye-tracking image capture unit.

26. The method of claim 25, further comprising:
determining, by a controller coupled to the eye-tracking image capture unit, an interpupillary distance of the user using the captured right eye image and the captured left eye image.

27. The method of claim 26, further comprising:
commanding, by the controller, movement of the first lens and the second lens to positions having a separation corresponding to the interpupillary distance.

28. The method of claim 25, further comprising:
determining, by a controller coupled to the eye-tracking image capture unit, a distance between a first internal visible marker image captured with the captured right eye image and a second internal visible marker image captured with the captured left eye image.

29. The method of claim 28, further comprising:
commanding, by the controller, movement of a first internal visible marker and a second internal visible marker to positions having a separation corresponding to a saved viewer optic spacing on a condition that the distance between the first internal visible marker image captured with the captured right eye image and the second internal visible marker image captured with captured left eye image is not equal to the saved viewer optic spacing.

30. The method of claim 25, wherein the first and second lenses are completely within a field of view of the eye-tracking image capture unit.
